(19) United States
(12) Patent Application Publication
(10) Pub. No.: US 2009/0138079 A1
(43) Pub. Date: May 28, 2009

(54) PROSTHETIC HEART VALVE FOR TRANSFEMORAL DELIVERY

(75) Inventors: Yosi Tuval, Netanya (IL); Igor Kovalsky, Givataim (IL)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(73) Assignee: VECTOR TECHNOLOGIES LTD., Netanya (IL)

(21) Appl. No.: 12/248,776

(22) Filed: Oct. 9, 2008

Related U.S. Application Data
(60) Provisional application No. 60/978,794, filed on Oct. 10, 2007.

(51) Int. Cl. A61F 2/24; A61B 19/00

(52) U.S. Cl. 623/2.11; 128/898

(57) ABSTRACT

Apparatus is provided that includes a valve prosthesis for attachment to a native valve complex of a subject. The prosthesis includes a support frame, which is shaped so as to define a plurality of axial support arches, which extend in a radially outward direction, and are configured to apply, regardless of a rotational orientation of the support frame with respect to the native valve complex, an upstream axial force to a downstream side of one or more native structures selected from the group consisting of: native leaflet tips of the native valve complex, and native valve commissures. The support frame is also shaped so as to define an upstream skirt, which is configured to apply a downstream axial force on an upstream side of the native valve complex. The prosthesis further includes a prosthetic heart valve, coupled to a portion of the support frame. Other embodiments are also described.

### CROSS-REFERENCE TO RELATED APPLICATIONS

The present application claims the benefit of: (a) U.S. Provisional Application No. 60/978,794, filed Oct. 10, 2007, entitled, “Prosthetic heart valve specially adapted for transfemoral delivery,” and (b) a US provisional application Ser. No. ______, filed Sep. 15, 2008, entitled, “Prosthetic heart valve for transfemoral delivery,” both of which are incorporated herein by reference in their entireties.

### FIELD OF THE INVENTION

The present invention relates generally to prosthetic heart valves, and specifically to prosthetic heart values configured for transfemoral delivery.

### DESCRIPTION OF THE RELATED ART

Aortic valve replacement in patients with severe valve disease is a common surgical procedure. The replacement is conventionally performed by open heart surgery, in which the heart is usually arrested and the patient is placed on a heart bypass machine. In recent years, prosthetic heart valves have been developed which are implanted using minimally invasive procedures such as transapical or percutaneous approaches. These methods include compressing the prosthesis radially to reduce its diameter, inserting the prosthesis into a delivery tool, such as a catheter, and advancing the delivery tool to the correct anatomical position in the heart. Once properly positioned, the prosthesis is deployed by radial expansion within the native valve annulus.

PCT Publication WO 05/002466 to Schwammenthal et al., relevant portions of which are incorporated herein by reference, describes prosthetic devices for treating aortic stenosis.

PCT Publication WO 06/070372 to Schwammenthal et al., relevant portions of which are incorporated herein by reference, describes a prosthetic device having a single flow field therethrough, adapted for implantation in a subject, and shaped so as to define a fluid inlet and a diverging section, distal to the fluid inlet.

US Patent Application Publication 2006/0149630 to Schwammenthal et al., relevant portions of which are incorporated herein by reference, describes a prosthetic device including a valve-orifice attachment member attachable to a valve in a blood vessel and including a fluid inlet, and a diverging member that extends from the fluid inlet, the diverging member including a proximal end near the fluid inlet and a distal end distanced from the proximal end. A distal portion of the diverging member has a larger cross-sectional area for fluid flow therethrough than a proximal portion thereof.

US Patent Application Publication 2004/0236411 to Sarac et al., relevant portions of which are incorporated herein by reference, describes a prosthetic valve for replacing a cardiac valve, which includes an expandable support member and at least two valve leaflets made of a first layer of biological material selected from peritoneal tissue, pleural tissue or pericardial tissue. A second layer of biological material is attached to the support member. The second layer is also made from peritoneal tissue, pleural tissue or pericardial tissue. The second layer includes a radially inwardly facing surface that defines a conduit for directing blood flow. The valve leaflets extend across the conduit to permit unidirectional flow of blood through the conduit. Methods for making and implanting the prosthetic valve are also described.

US Patent Application Publication 2006/0259136 to Nguyen et al., relevant portions of which are incorporated herein by reference, describes a heart valve prosthesis having a self-expanding multi-level frame that supports a valve body comprising a skirt and plurality of coapting leaflets. The frame transitions between a contracted delivery configuration that enables percutaneous transluminal delivery, and an expanded deployed configuration having an asymmetric hourglass shape. The valve body skirt and leaflets are constructed so that the center of coaptation may be selected to reduce horizontal forces applied to the commissures of the valve, and to efficiently distribute and transmit forces along the leaflets and to the frame. Alternatively, the valve body may be used as a surgically implantable replacement valve prosthesis.

The following patents and patent application publications, relevant portions of which are incorporated herein by reference, are of interest:

US Patent Application Publication 2005-0197695 to Stacchino et al.

U.S. Pat. No. 6,312,465 to Griffin et al.

U.S. Pat. No. 5,908,451 to Yeo

U.S. Pat. No. 5,344,442 to Deac

U.S. Pat. No. 5,354,330 to Hanson

US Patent Application Publication 2004-0260389 to Case et al.

U.S. Pat. No. 6,730,118 to Spencer et al.

U.S. Pat. No. 7,018,406 to Seguin et al.

U.S. Pat. No. 7,018,408 to Bailey et al.

U.S. Pat. No. 6,458,153 and US Patent Application Publication 2003-0023300 to Bailey et al.

US Patent Application Publication 2004-0186563 to Lobbi

US Patent Application Publication 2003-0130729 to Paniagua et al.

US Patent Application Publication 2004-0236411 to Sarac et al.

US Patent Application Publication 2005-0075720 to Nguyen et al.

US Patent Application Publication 2006-0058872 to Salahieh et al.

US Patent Application Publication 2005-0137688 to Salahieh et al.

US Patent Application Publication 2005-0137690 to Salahieh et al.

US Patent Application Publication 2005-0137691 to Salahieh et al.

US Patent Application Publication 2005-0143809 to Salahieh et al.

US Patent Application Publication 2005-0182483 to Osborne et al.

US Patent Application Publication 2005-0137695 to Salahieh et al.

US Patent Application Publication 2005-0240200 to Bergheim

US Patent Application Publication 2006-0025857 to Bergheim et al.

US Patent Application Publication 2006-0025855 to Lashinski et al.

US Patent Application Publication 2006-0047338 to Jenson et al.

US Patent Application Publication 2006-0052867 to Revuelta et al.

US Patent Application Publication 2006-0074485 to Realyvasquez

US Patent Application Publication 2003-0149478 to Figulla et al.

U.S. Pat. No. 7,137,184 to Schreck

U.S. Pat. No. 6,296,662 to Caffey

U.S. Pat. No. 6,558,418 to Carpentier et al.

U.S. Pat. No. 7,267,686 to DiMatteo et al.

### SUMMARY OF THE INVENTION

In some embodiments of the present invention, a prosthetic heart valve prosthesis comprises a collapsible support frame and a prosthetic valve. The support frame is typically shaped so as to define three commissural posts to which the prosthetic valve is coupled, and an upstream skirt that is configured to apply an axial force in a downstream direction on an upstream side of the native annulus and left ventricular outflow tract (LVOT). A portion of cells of the support frame are shaped so as to define a plurality of outwardly-extending short axial support arches, which extend in a radially outward direction (away from the central longitudinal axis of the prosthesis). The shape of the support frame allows the valve prosthesis to be implanted such that an upstream section of the prosthesis is positioned upstream to the native annulus of the patient, while the axial support arches protrude over the tips of the native leaflets, and collectively define an outer diameter that is greater than the diameter of the tips of the native leaflets. The axial support arches are distributed around the circumference of the frame such that, depending on the rotational orientation of the valve prosthesis, the arches engage and rest against either a native valve commissure (riding astride the commissure) or a leaflet tip, such that the valve prosthesis is anchored axially regardless of the rotational orientation of the prosthesis. The axial support arches are sized so as to not extend to the floors of the aortic sinuses.

The support frame applies an axial force to the native valve complex from below and above the complex, anchoring the valve prosthesis in place, and inhibiting migration of the prosthetic valve both upstream and downstream. This configuration also allows the valve prosthesis to apply outward radial force to the native valve, in order to prevent blood leakage between the valve prosthesis and the native valve. Such outward radial force typically does not substantially aid with fixation of the valve prosthesis at the native valve complex.

There is therefore provided, in accordance with an embodiment of the present invention, apparatus including a valve prosthesis for attachment to a native valve complex of a subject, the prosthesis including:

a support frame, which is shaped so as to define:

a plurality of axial support arches, which extend in a radially outward direction, and are configured to apply, regardless of a rotational orientation of the support frame with respect to the native valve complex, an upstream axial force to a downstream side of one or more native structures selected from the group consisting of: native leaflet tips of the native valve complex, and native valve commissures, and

an upstream skirt, which is configured to apply a downstream axial force on an upstream side of the native valve complex; and

a prosthetic heart valve, coupled to a portion of the support frame.

For some applications, the axial support arches extend in an upstream and radially outward direction. For other applications, the axial support arches extend radially outward in a direction orthogonal to a central longitudinal axis of the prosthesis. Typically, the axial support arches protrude radially outward over the native leaflet tips. Typically, the axial support arches are sized so as to not extend to floors of aortic sinuses of the native valve complex.

For some applications, the support frame is configured to assume a radially collapsed position for delivery to the native valve complex, and a radially expanded position upon implantation at the native valve complex.

For some applications, the support frame is shaped so as to define a multiple of three of the axial support arches. For some applications, a longitudinal distance from an upstream-most portion of each of the axial support arches to a downstream-most portion of the axial support arch is no more than 15 mm.

For some applications, the support frame is shaped so as to define a plurality of commissural posts, and at least one end of each of the axial support arches is coupled to one of the commissural posts.

For some applications, the axial support arches extend in the upstream and radially outward direction at an angle of between 110 and 30 degrees with respect to a central longitudinal axis of the prosthesis.

For some applications, the valve prosthesis is configured to not radially squeeze native leaflets of the native valve complex between any elements of the valve prosthesis. Typically, the valve prosthesis is configured such that the upstream and downstream axial forces together anchor the valve prosthesis to the native valve complex.

For some applications, the prosthetic valve is coupled to a downstream section of the support frame, the support frame is shaped so as to define a throat section longitudinally between the upstream skirt and the downstream section, a cross-sectional area of the valve prosthesis gradually decreases from the upstream skirt to the throat section, and gradually increases from the throat section to the downstream section, and a cross-sectional area of the throat section is less than a cross-sectional area of an aortic annulus of the native valve complex.

There is further provided, in accordance with an embodiment of the present invention, a method including:

providing a valve prosthesis, which includes (a) a support frame, which is shaped so as to define (i) a plurality of axial support arches, which extend in a radially outward direction, and (ii) an upstream skirt, and (b) a prosthetic heart valve, coupled to a portion of the support frame; and

positioning the valve prosthesis at a native valve complex of a subject, such that:

the axial support arches apply, regardless of a rotational orientation of the support frame with respect to the native valve complex, an upstream axial force to a downstream side of one or more native structures selected from the group consisting of: native leaflet tips of the native valve complex, and native valve commissures, and

the upstream skirt applies a downstream axial force on an upstream side of the native valve complex.

For some applications, providing the valve prosthesis includes providing the valve prosthesis such that the axial support arches extend in an upstream and radially outward direction. For other applications, providing the valve prosthesis includes providing the valve prosthesis such that the axial support arches extend radially outward in a direction orthogonal to a central longitudinal axis of the prosthesis.

For some applications, positioning the valve prosthesis does not include rotationally aligning the valve prosthesis with respect to the native valve complex.

For some applications, positioning the valve prosthesis includes positioning the valve prosthesis such that the axial support arches protrude radially outward over the native leaflet tips. For some applications, positioning the valve prosthesis includes positioning the valve prosthesis such that the axial support arches do not extend to floors of aortic sinuses of the native valve complex.

For some applications, positioning the valve prosthesis includes: radially collapsing the support frame prior to delivery of the valve prosthesis to the native valve complex; transluminally delivering the valve prosthesis to a vicinity of the native valve complex while the support frame is radially collapsed; and radially expanding the support frame at the native valve complex.

Typically, positioning the valve prosthesis includes positioning the valve prosthesis such that the upstream and downstream axial forces together anchor the valve prosthesis to the native valve complex.

The present invention will be more fully understood from the following detailed description of embodiments thereof, taken together with the drawings.

### BRIEF DESCRIPTION OF THE DRAWINGS

FIGS. 1A and 1B are schematic illustrations of a valve prosthesis, in accordance with an embodiment of the present invention;

FIGS. 2A and 2B are schematic illustrations of a delivery system for delivering the valve prosthesis of FIGS. 1A and 1B to a target site and implanting the prosthesis at the site, in accordance with an embodiment of the present invention;

FIG. 3 is a schematic cross-sectional illustration of a front end of a catheter of the delivery system of FIG. 2, in accordance with an embodiment of the present invention;

FIGS. 4A-L schematically illustrate a procedure for implanting the valve prosthesis of FIGS. 1A and 1B using the delivery system of FIG. 2, in accordance with an embodiment of the present invention;

FIGS. 5A-C are schematic illustrations of three different possible rotational orientations of the valve prosthesis of FIGS. 1A and 1B with respect to the native valve upon deployment, in accordance with an embodiment of the present invention;

FIGS. 6A-B are schematic illustrations of a catheter tube, in accordance with an embodiment of the present invention; and

FIG. 7 is a schematic illustration of a shaped balloon, in accordance with an embodiment of the present invention.

### DETAILED DESCRIPTION OF THE PREFERRED EMBODIMENT

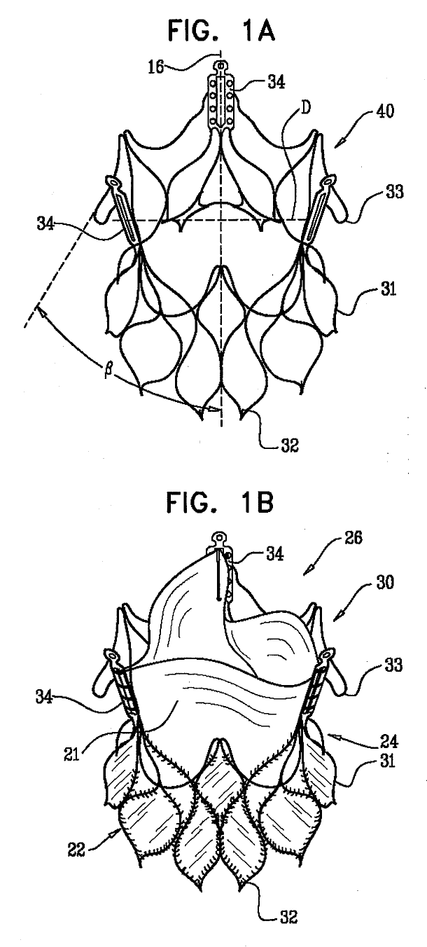

FIGS. 1A and 1B are schematic illustrations of a valve prosthesis 30, in accordance with an embodiment of the present invention. FIG. 1B shows the prosthesis including a prosthetic valve 21 and a skirt 31, as described below, while FIG. 1A shows the prosthesis without these elements for clarity of illustration. Valve prosthesis 30 comprises a collapsible support frame 40, which typically comprises exactly three commissural posts 34, arranged circumferentially around a central longitudinal axis 16 of valve prosthesis 30. Valve prosthesis 30 further comprises prosthetic downstream valve 21 coupled to commissural posts 34. Valve 21 typically comprises a pliant material. The pliant material is configured to collapse inwardly (i.e., towards central longitudinal axis 16) during diastole, in order to inhibit retrograde blood flow, and to open outwardly during systole, to allow blood flow through the prosthesis.

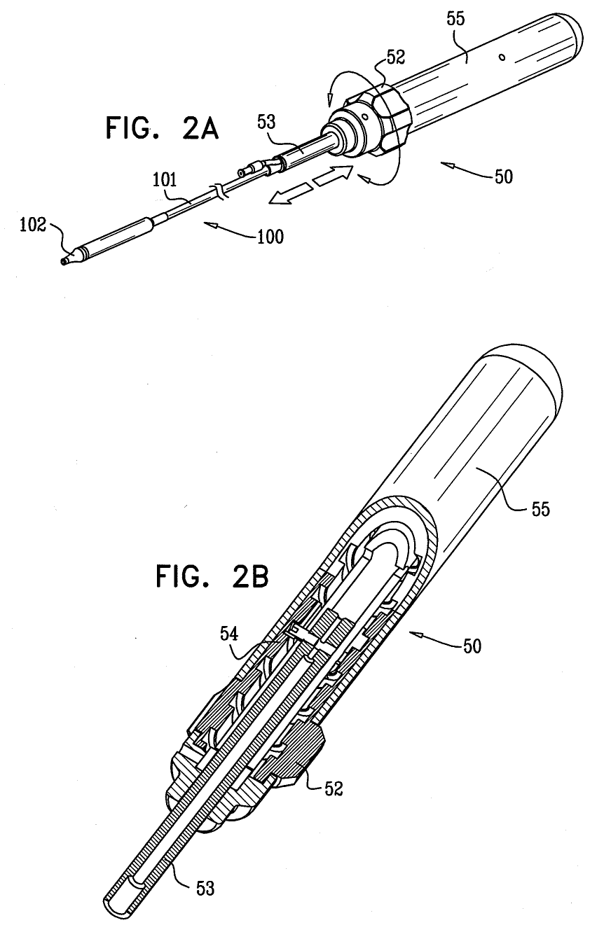

Valve prosthesis 30 is configured to be implanted in a native diseased valve of a patient, such as a native stenotic aortic or pulmonary valve, using a minimally-invasive approach, such as a beating heart endovascular retrograde transaortic, e.g. transfemoral, procedure. Support frame 40 is typically collapsed or crimped so that its diameter is reduced in order to facilitate loading onto a catheter or cannula for delivery to the native valve site during a minimally-invasive delivery procedure, as described hereinbelow with reference to FIGS. 2, 3, and 4A-L. Support frame 40 is configured such that application of radial forces thereon radially compress the frame, reducing the frame's outer diameter. Upon removal of the radial forces, the frame assumes its earlier diameter and shape. The prosthesis, while the frame is in its compressed state, is loaded into a tube sufficiently small to allow transluminal delivery to the patient's native valve site. Support frame 40 comprises a suitable material that allows mechanical deformations associated with collapsing and expansion of valve prosthesis 30, such as, but not limited to, a superelastic material, such as nitinol, or a stainless steel alloy (e.g., AISI 316).

Support frame 40 is typically shaped to define an upstream section 22, a throat section 24, and a downstream section 26 (as indicated in FIG. 1B). The cross-sectional area of upstream section 22 gradually decreases from an upstream end thereof to a downstream end adjacent to throat section 24. The cross-sectional area of throat section 24 is typically less than that of the aortic annulus of the intended patient. The cross-sectional area of downstream section 26 gradually increases to an area greater than that of throat section 24. Thus the cross-sectional areas of both the upstream and downstream sections are greater than that of the throat section. Throat section 24 is configured to be placed within the leaflet section of the native valve, slightly above the aortic annulus at the ventriculo-aortic border, such that downstream section 26 is located in the aorta, such as in the aortic sinuses. Typically, throat section 24 is configured to exert an outward radial force against the native leaflets, in order to prevent blood leakage between the valve prosthesis and the native valve. Such outward radial force typically does not substantially aid with fixation of the valve prosthesis at the native valve complex, and typically does not radially squeeze the native leaflets between the throat section any other elements of valve prosthesis 30 (including axial support arches 33).

Typically, support frame is elastic, and is shaped so as to define a plurality of collapsible cells. For example, the support frame may be fabricated by cutting a solid tube. The cells may be diamond-shaped, parallelogram-shaped, or otherwise shaped to be conducive to collapsing the frame. Downstream section 26 is typically shaped so as to define bulging upstream skirt 31, which is configured to apply a downstream axial force directed toward the ascending aorta. Optionally, skirt 31 is shaped so as to define one or more barbs 32 positioned circumferentially such that the barbs pierce the native vale annulus in order to provide better anchoring. Typically, valve prosthesis 30 further comprises a skirt covering 35 which is coupled to upstream skirt 31, such as by sewing the covering within the skirt (configuration shown in FIG. 1B) or around the skirt (configuration not shown). Skirt covering 35 may comprise, for example, polyester or a processed biological material, such as pericardium. Support frame 40 thus defines a central structured body for flow passage that terminates in an upstream direction in a flared inlet (upstream skirt 31) that is configured to be seated within an LVOT immediately below an aortic annulus/aortic valve.

Typically, a portion of the cells of support frame 40 are shaped to define a plurality of outwardly-extending short axial support arches 33, which extend in a radially outward direction (away from central longitudinal axis 16). For some applications, axial support arches 33 also extend in an upstream direction (as shown in the figures), while for other applications, the axial support arches extend in a downstream direction, or in a direction orthogonal to central longitudinal axis 16 (configurations not shown). Axial support arches 33 are distributed around the circumference of the frame at a predetermined height from the upstream end of the frame, and may be either evenly (as shown in FIGS. 1A and 1B) or unevenly distributed (not shown in the figures) around the circumference. Support frame 40 typically is shaped to define at least three axial support arches 33, such as greater than three arches. For some applications, the number of support arches is a multiple of three, such as six (as shown in FIGS. 1A and 1B). Typically, a longitudinal distance from an upstream-most portion of each axial support arch 33 to a downstream-most portion of the axial support arch is no more than 15 mm, such as no more than 12 mm, and is at least 1 mm, such as at least 8 mm.

For some applications, as shown in FIGS. 1A and 1B, at least one end of each axial support arch 33 is coupled to one of commissural posts 34. The other end of each axial support arch 33 may be coupled to (a) another more upstream cell of the prosthesis, as shown in FIGS. 1A and 1B, (b) another of commissural posts 34 (such as for applications in which the prosthesis has exactly three axial support arches 33) (configuration not shown), or (c) to an end of another of axial support arches 33 (configuration not shown).

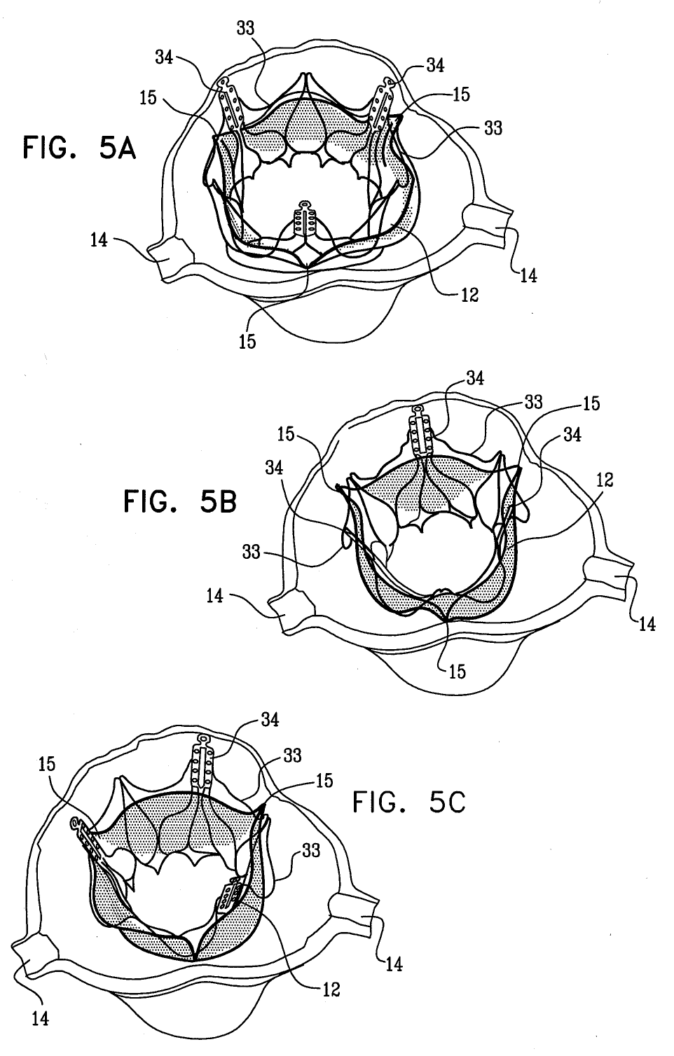

The shape of support frame 40 allows valve prosthesis 30 to be implanted such that upstream section 22 is positioned upstream to the native annulus of the patient, while axial support arches 33 protrude over the tips of the native leaflets, and collectively define an outer diameter D that is greater than the diameter of the tips of the native leaflets. Axial support arches 33 flare out laterally in an upstream direction during deployment at an angle β with central longitudinal axis 16 of valve prosthesis 30. Angle β is typically between about 170 and about 10 degrees, such as between about 110 and about 30 degrees. Axial support arches 33 are radially distributed around the frame such that, depending on the rotational orientation of valve prosthesis 30, the axial support arches engage and rest against either a native valve commissure (riding astride the commissure) or a leaflet tip, such that the valve prosthesis is anchored axially regardless of the rotational orientation of the prosthesis, as described in more detail hereinbelow with reference to FIGS. 5A-C. Axial support arches 33 are sized so as to not extend to the floors of the aortic sinuses. This configuration applies an axial force to the native valve complex from below and above the complex, anchoring valve prosthesis 30 in place, and inhibiting migration of the prosthetic valve both upstream and downstream. This configuration also allows the valve prosthesis to apply outward radial force to the native valve, in order to prevent blood leakage between the valve prosthesis and the native valve. Such outward radial force typically does not substantially aid with fixation of the valve prosthesis at the native valve complex, and typically does not radially squeeze the native leaflets between any elements of valve prosthesis 30 (including axial support arches 33).

Although exactly three commissural posts 34 are shown in the figures, for some applications valve prosthesis 30 comprises fewer or more posts 34, such as two posts 34, or four or more posts 34. It is noted that approximately 90% of humans have exactly three aortic sinuses. The three posts provided in most embodiments correspond to these three aortic sinuses. For implantation in the approximately 10% of patients that have exactly two aortic sinuses, prosthesis 30 may include exactly two posts.

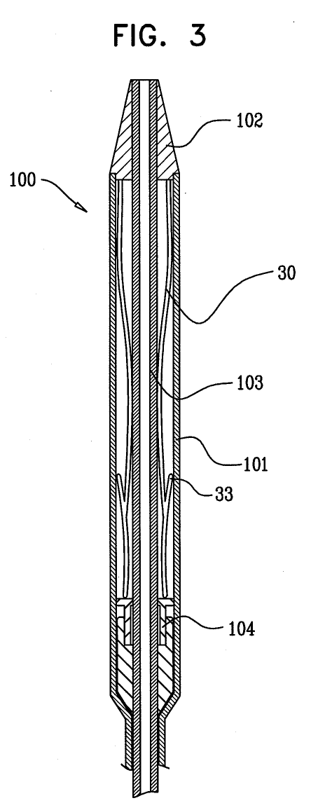

FIG. 2A is a schematic illustration of a delivery system 50 for delivering valve prosthesis 30 to a target site and implanting the prosthesis at the site, and FIG. 2B is a schematic cross-sectional illustration of a proximal portion of the delivery system, in accordance with an embodiment of the present invention. FIG. 3 is a schematic cross-sectional illustration of a front end of catheter 100, in accordance with an embodiment of the present invention. Delivery system 50 comprises a catheter 100, which comprises an inner neutral tube 103 (shown in FIG. 3) which is concentric with an outer tube 101. The diameter of outer tube 101 typically varies along catheter 100. Neutral tube 103 is fixed so that it does not move when outer tube 101 is moved backwards by turning a knob 52. A tip 102 of catheter 100 is located at an upstream end of neutral tube 103, such that outer tube 101 abuts against tip 102 when catheter 100 is in a closed position, as shown in FIG. 2A. Delivery system 50 is used to effect the release of valve prosthesis 30 (the prosthesis is not shown in FIGS. 2A and 2B) by moving outer tube 101 with respect to neutral tube 103. Delivery system 50 further comprises an outer tube holder 53 set within delivery body 55. To open the catheter, knob 52 is turned clockwise, causing a screw 54 to turn the circular motion into a linear motion, thereby causing outer tube holder 53 to move backward. The backward motion of outer tube holder 53 causes prosthetic valve 30 to be gradually exposed until it is free of outer tube 101.

Valve prosthesis 30 is shown in FIG. 3 within the catheter in the prosthesis's compressed state, held in a valve holder 104 and compressed between neutral tube 103 and outer tube 101. The catheter is shown in its closed state, such that the upstream end of outer tube 101 rests against the downstream end of tip 102.

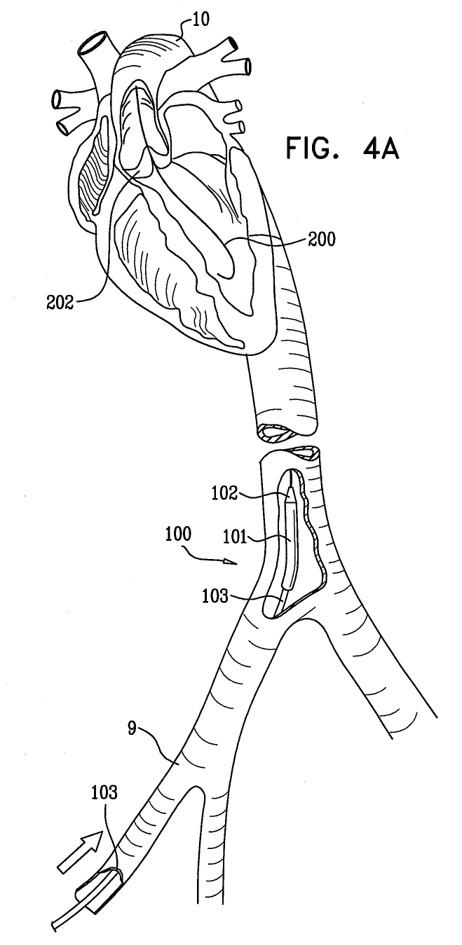

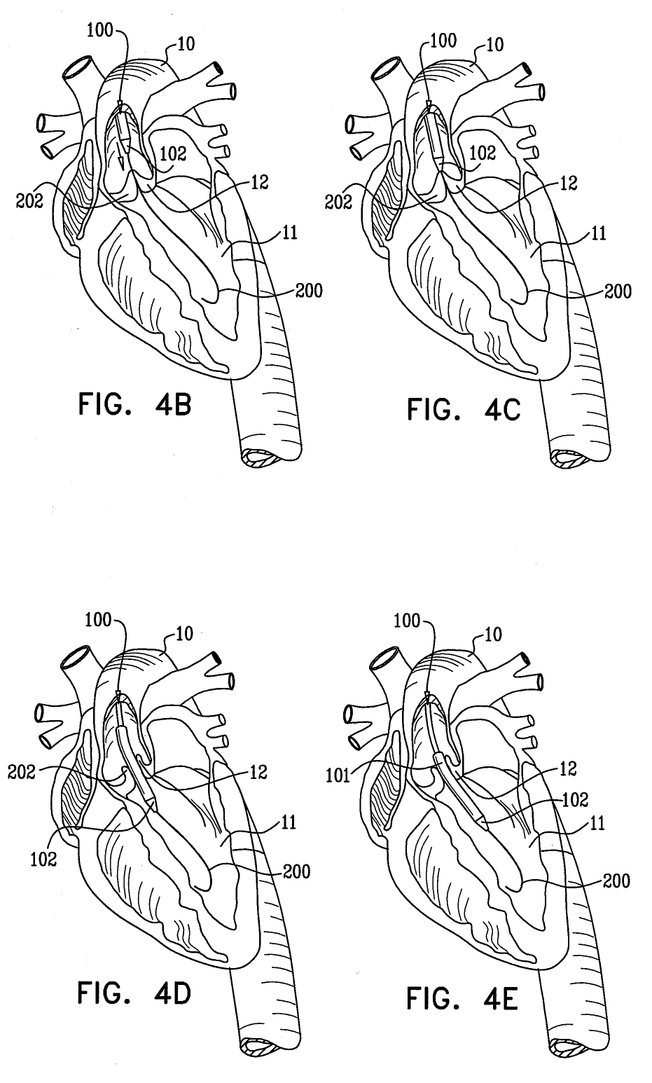

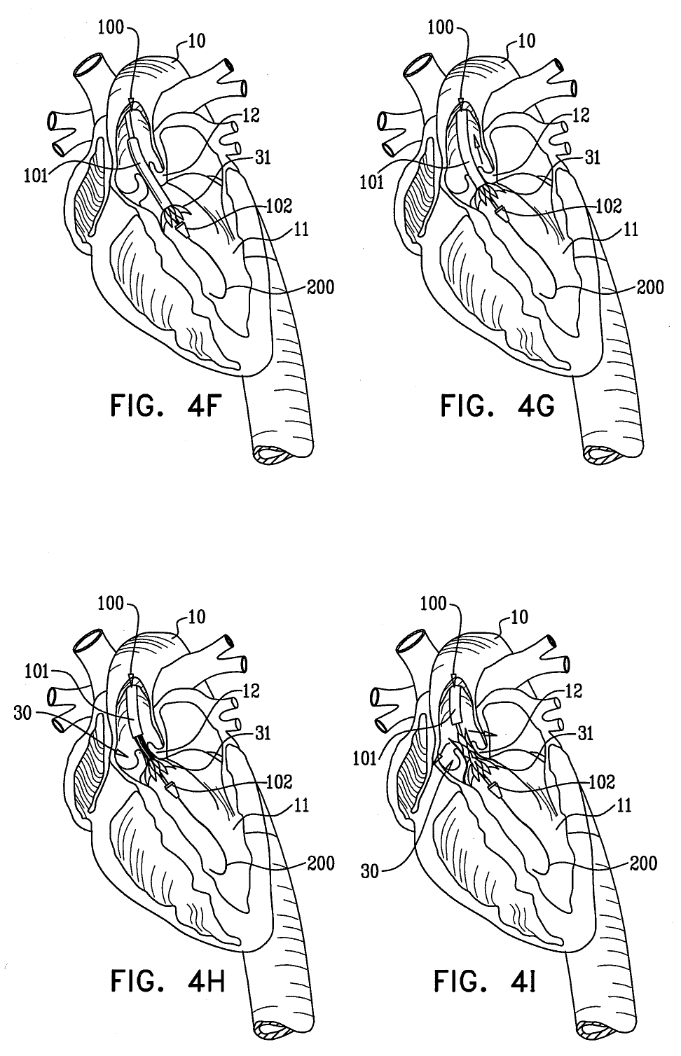

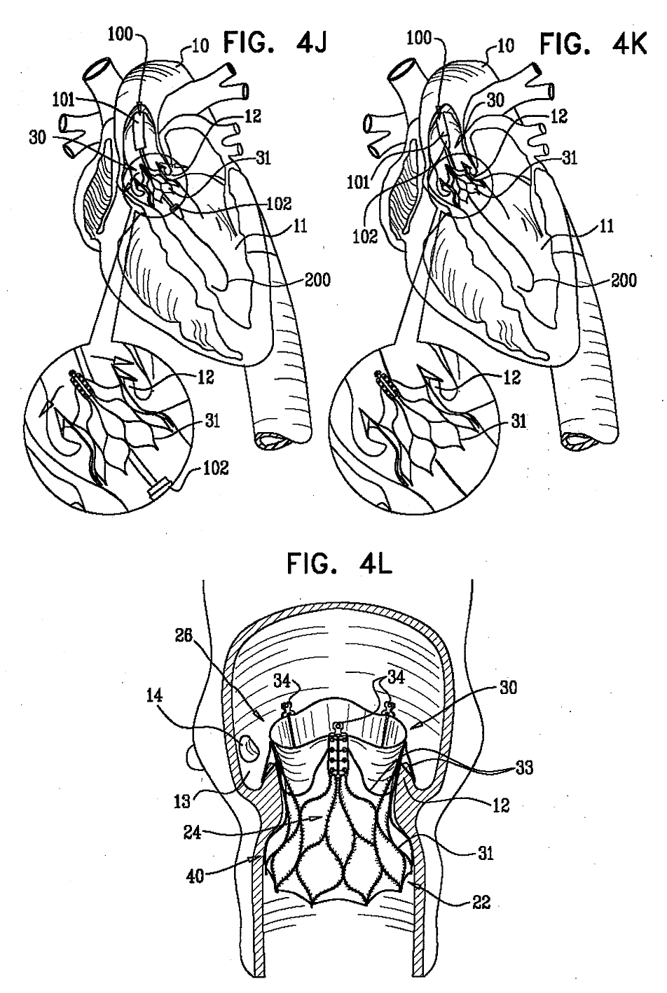

FIGS. 4A-L schematically illustrate a procedure for implanting valve prosthesis 30 using delivery system 50, in accordance with an embodiment of the present invention. Although these figures show the implantation of the prosthesis in an aortic position, these techniques, as appropriately modified, may also be used to implant the prosthesis in other locations, such as in a pulmonary valve.

As shown in FIG. 4A, delivery catheter 100 is inserted into a body lumen 9. For some applications, body lumen 9 is a femoral artery. The catheter is inserted into body lumen 9, and is guided over a guidewire 200 through the ascending aorta and over an aortic arch 10. Optionally, a stenotic aortic valve 202 is partially dilated to about 15-20 mm (e.g., about 16 mm), typically using a standard valvuloplasty balloon catheter.

As shown in FIG. 4B, catheter 100, which rides over guidewire 200, is passed over aortic arch 10 towards native aortic valve 202. The tip of guidewire 200 passes into a left ventricle 11.

As shown in FIG. 4C, catheter tip 102 is advanced toward the junction of native aortic valve leaflets 12 towards left ventricle 11, while the catheter continues to ride over the guidewire.

As shown in FIG. 4D, catheter tip 102 is brought past native aortic valve leaflets 12 into left ventricle 11. Outer tube 101 of catheter 100 is located between native aortic leaflets 12.

As shown in FIG. 4E, catheter tip 102 is further advanced, past aortic leaflets 12 and deeper into left ventricle 11. Outer tube 101 of catheter 100 is still located between native aortic leaflets 12.

As shown in FIG. 4F, outer tube 101 is withdrawn a predetermined distance to expose upstream skirt 31 of valve prosthesis 30. Outer tube 101 moves with respect to inner tube 103, such that valve prosthesis 30 and inner tube 103 are partially exposed from the catheter. Skirt 31 is positioned within left ventricle 11. At this point during the implantation procedure, skirt 31 may not yet have come in contact with the ventricular side of native aortic leaflets 12.

As shown in FIG. 4G, catheter 100 is withdrawn until skirt 31 abuts firmly against the ventricular side of the aortic annulus and/or aortic valve leaflets 12. If provided, barbs 32 may pierce the native annulus, or may rest against the ventricular side of the valve complex.

As shown in FIG. 4H, outer tube 101 is further withdrawn until the tube is located just upstream of the ends of commissural posts 34 of valve prosthesis 30, such that the commissural posts are still held firmly by outer tube 101.

FIG. 4I shows valve prosthesis 30 immediately upon release from outer tube 101. Support frame 40, which is typically superelastic, rapidly expands to its fully opened position, pushing native valve leaflets 12 radially outward.

FIG. 4J shows the opening of valve prosthesis 30 to its fully expanded shape. Prosthetic valve 30 is thus released with the outer tube being moved in only one direction during the entire procedure, which facilitates the implantation procedure significantly.

FIG. 4K shows catheter 100 in its closed position, with outer tube 101 resting firmly against catheter tip 102. Catheter 100 is withdrawn over the aortic arch, still riding on guidewire 200.

FIG. 4L is a schematic illustration of prosthetic valve 30 in the aortic position, in accordance with an embodiment of the present invention. Skirt 31 is positioned within ventricle 11 such that throat section 24 of support frame 40 is located in close proximity to the native annulus between native leaflets 12. Commissural posts 34 of valve prosthesis 30 generally define a diverging shape, and are located on the arterial side of the native valve. Native valve leaflets 12 generally follow the contour of valve prosthesis 30. Axial support arches 33 protrude over the tips of native leaflets 12, so that they provide axial support to prevent device embolism into ventricle 11 through native leaflets 12 during the cardiac cycle. It is noted that in the configuration shown, valve prosthesis 30 does not include barbs 32, described hereinabove with reference to FIGS. 1A and 1B.

FIGS. 5A-C are schematic illustrations of three different possible rotational orientations of valve prosthesis 30 with respect to the native valve upon deployment, in accordance with an embodiment of the present invention. All of these rotational orientations, as well as intermediate rotational orientations not shown, provide proper axial fixation of the valve prosthesis. For clarity of illustration, in FIGS. 5A-C only support frame 40 of the valve prosthesis is shown, without prosthetic downstream valve 21 or skirt covering 35 of skirt 31. The valve prosthesis is deployed within the aortic root, which includes aortic sinuses, coronary ostia 14, and native valve commissures 15. Upon implantation, valve prosthesis 30 provides axial anchoring on both sides (ventricular and arterial) of the native valve annulus. Skirt 31 extends radially below the annulus, providing an axial force applied in the arterial (downstream) direction to the underside of the annulus, while axial support arches 33 exert an axial force in the ventricular (upstream) direction by resting against the tips of native leaflets 12 or native commissures 15.

FIG. 5A shows a first possible rotational orientation of valve prosthesis 30, in which commissural posts 34 of the prosthesis are essentially aligned with native commissures 15, allowing axial support arches 33 to rest against the tips of native leaflets 12.

FIG. 5B shows another possible rotational orientation of prosthetic valve 30 within the native valve complex, in which commissural posts 34 of the prosthesis are positioned at a rotational offset of about 60 degrees with respect to native commissures 15, with axial support arches 33 extending over the tips of native leaflets 12. As can be seen in FIG. 5B, axial support arches 33 provide axial anchoring, regardless of the rotational orientation of the prosthesis with respect to the native valve. Therefore, axial support arches 33, which are circumferentially distributed around prosthetic valve 30, obviate the need to rotationally align prosthetic valve 30 with any anatomical feature of the native valve complex, since axial support arches 33 are generally guaranteed to be located between native commissures 15, or riding astride the native commissures 15.

FIG. 5C shows yet another possible rotational orientation of prosthetic valve 30 within the native valve complex upon deployment, in which commissural posts 34 of the prosthesis are offset with respect to native valve commissures 15 by about 30 degrees. Even in this particular rotationally asymmetric position, axial support arches 33 engage the tips of native leaflets 12, or native valve commissures 15, effectively applying an upstream axial force to the native structure, obviating the need for deliberate rotational positioning of prosthetic valve 30 during the implantation process.

For some applications, prosthesis 30 is implanted using some of the techniques described with reference to FIGS. 9A-G in U.S. application Ser. No. 12/050,628, filed Mar. 18, 2008, entitled, “Valve suturing and implantation procedures,” which is incorporated herein by reference.

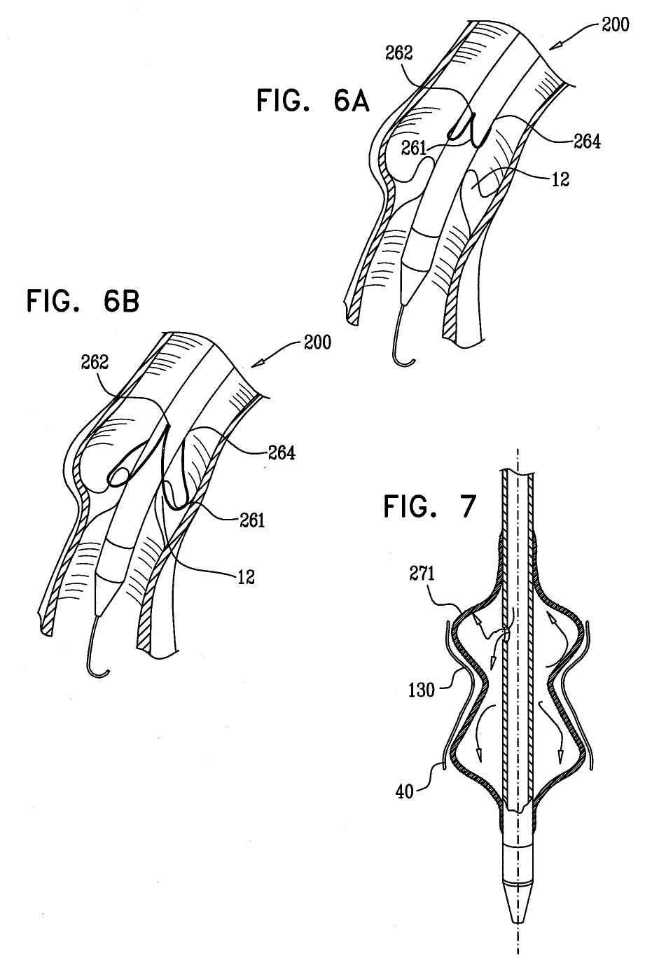

FIGS. 6A and 6B are schematic illustrations of a catheter tube 200, in accordance with an embodiment of the present invention. Catheter tube 200 comprises feelers 261 which are configured to enter the aortic sinuses and touch the floors of the sinuses, thereby aligning themselves with the sinuses. This alignment of the feelers with the sinuses aligns the delivery catheter both rotationally and axially with respect to the sinuses. A valve prosthesis compressed at least partially within catheter tube 200 is aligned with the feelers, such that when the prosthesis is released from the catheter tube, elements of the prosthesis, such as commissural posts, are properly rotationally aligned with elements of the native valve complex, such as the native valve commissures, and are at the proper axial position with respect to the native valve complex.

Feelers 261 are initially partially retracted into an outer tube 264, such that only a small portion of the feelers extend out through slits 262 defined by the outer tube, and rest against an outer surface of the outer tube, as shown in FIG. 6A. During an implantation procedure, the feelers are extended out of the outer tube, as shown in FIG. 6B. Slits 262 may be arrange circumferentially around the catheter tube. Feelers 261 may be extended and retracted by the physician, so that the feelers are in a retracted position while the catheter is advanced through the vasculature, and are extended before the delicate placement stage of the implantation procedure.

FIG. 7 is a schematic illustration of a shaped balloon 271, in accordance with an embodiment of the present invention. The balloon is used to plastically deform support structure 40 of valve prosthesis 30 or 130, and to give the structure a non-cylindrical shape. In this embodiment, support structure 40 or 140 may comprise a stainless steel alloy which is plastically deformed during crimping, thereby reducing the valve diameter, and mounted onto the balloon prior to implantation. When the delivery catheter is in place in the patient, shaped balloon 271 is used to open the crimped prosthesis into place, and to give it a non-cylindrical shape.

In the present patent application, including in the claims, the word “downstream” means near or toward the direction in which the blood flow is moving, and “upstream” means the opposite direction. For embodiments in which the valve prosthesis is implanted at the aortic valve, the aorta is downstream and the ventricle is upstream. As used in the present patent application, including in the claims, the “native valve complex” includes the native semilunar valve leaflets, the annulus of the valve, the subvalvular tissue on the ventricular side, and the lower half of the semilunar sinuses. As used in the present application, including in the claims, a “native semilunar valve” is to be understood as including: (a) native semilunar valves that include their native leaflets, and (b) native semilunar valves, the native leaflets of which have been surgically excised or are otherwise absent.

For some applications, techniques described herein are performed in combination with techniques described in a US provisional patent application filed on even date herewith, entitled, “Prosthetic heart valve having identifiers for aiding in radiographic positioning,” which is assigned to the assignee of the present application and is incorporated herein by reference.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
U.S. patent application Ser. No. 11/024,908, filed Dec. 30, 2004, entitled, “Fluid flow prosthetic device,” which issued as U.S. Pat. No. 7,201,772;
International Patent Application PCT/IL2005/001399, filed Dec. 29, 2005, entitled, “Fluid flow prosthetic device,” which published as PCT Publication WO 06/070372;
International Patent Application PCT/IL2004/000601, filed Jul. 6, 2004, entitled, “Implantable prosthetic devices particularly for transarterial delivery in the treatment of aortic stenosis, and methods of implanting such devices,” which published as PCT Publication WO 05/002466, and U.S. patent application Ser. No. 10/563,384, filed Apr. 20, 2006, in the national stage thereof, which published as US Patent Application Publication 2006-0259134;
U.S. Provisional Application 60/845,728, filed Sep. 19, 2006, entitled, “Fixation member for valve”;
U.S. Provisional Application 60/852,435, filed Oct. 16, 2006, entitled, “Transapical delivery system with ventriculo-arterial overflow bypass”;
U.S. application Ser. No. 11/728,253, filed Mar. 23, 2007, entitled, “Valve prosthesis fixation techniques using sandwiching”, which published as US Patent Application Publication 2008-0071363;
International Patent Application PCT/IL2007/001237, filed Oct. 16, 2007, entitled, “Transapical delivery system with ventriculo-arterial overflow bypass,” which published as PCT Publication WO 2008/047354;
U.S. application Ser. No. 12/050,628, filed Mar. 18, 2008, entitled, “Valve suturing and implantation procedures”; and/or
a US provisional application filed Sep. 15, 2008, entitled, “Prosthetic heart valve having identifiers for aiding in radiographic positioning.”

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

It is understood that the examples and embodiments described herein are for illustrative purposes only and that various modifications or changes in light thereof will be suggested to persons skilled in the art and are to be included within the spirit and purview of this application and scope of any appended claims. All figures, publications, patents, and patent applications cited herein are hereby incorporated by reference in their entirety for all purposes.

What is claimed is:

1. Apparatus comprising a valve prosthesis for attachment to a native valve complex of a subject, the prosthesis comprising:
a support frame, which is shaped so as to define:
a plurality of axial support arches, which extend in a radially outward direction, and are configured to apply, regardless of a rotational orientation of the support frame with respect to the native valve complex, an upstream axial force to a downstream side of one or more native structures selected from the group consisting of: native leaflet tips of the native valve complex, and native valve commissures, and
an upstream skirt, which is configured to apply a downstream axial force on an upstream side of the native valve complex; and
a prosthetic heart valve, coupled to a portion of the support frame.

2. The apparatus according to claim 1, wherein the axial support arches extend in an upstream and radially outward direction.

3. The apparatus according to claim 1, wherein the axial support arches extend radially outward in a direction orthogonal to a central longitudinal axis of the prosthesis.

4. The apparatus according to claim 1, wherein the axial support arches protrude radially outward over the native leaflet tips.

5. The apparatus according to claim 1, wherein the axial support arches are sized so as to not extend to floors of aortic sinuses of the native valve complex.

6. The apparatus according to claim 1, wherein the support frame is configured to assume a radially collapsed position for delivery to the native valve complex, and a radially expanded position upon implantation at the native valve complex.

7. The apparatus according to claim 1, wherein the support frame is shaped so as to define a multiple of three of the axial support arches.

8. The apparatus according to claim 1, wherein a longitudinal distance from an upstream-most portion of each of the axial support arches to a downstream-most portion of the axial support arch is no more than 15 mm.

9. The apparatus according to claim 1, wherein the support frame is shaped so as to define a plurality of commissural posts, and wherein at least one end of each of the axial support arches is coupled to one of the commissural posts.

10. The apparatus according to claim 1, wherein the axial support arches extend in the upstream and radially outward direction at an angle of between 110 and 30 degrees with respect to a central longitudinal axis of the prosthesis.

11. The apparatus according to claim 1, wherein the valve prosthesis is configured to not radially squeeze native leaflets of the native valve complex between any elements of the valve prosthesis.

12. The apparatus according to claim 1, wherein the valve prosthesis is configured such that the upstream and downstream axial forces together anchor the valve prosthesis to the native valve complex.

13. The apparatus according to claim 1,
wherein the prosthetic valve is coupled to a downstream section of the support frame,
wherein the support frame is shaped so as to define a throat section longitudinally between the upstream skirt and the downstream section,
wherein a cross-sectional area of the valve prosthesis gradually decreases from the upstream skirt to the throat section, and gradually increases from the throat section to the downstream section, and
wherein a cross-sectional area of the throat section is less than a cross-sectional area of an aortic annulus of the native valve complex.

14. A method comprising:
providing a valve prosthesis, which includes (a) a support frame, which is shaped so as to define (i) a plurality of axial support arches, which extend in a radially outward direction, and (ii) an upstream skirt, and (b) a prosthetic heart valve, coupled to a portion of the support frame; and
positioning the valve prosthesis at a native valve complex of a subject, such that:
the axial support arches apply, regardless of a rotational orientation of the support frame with respect to the native valve complex, an upstream axial force to a downstream side of one or more native structures selected from the group consisting of: native leaflet tips of the native valve complex, and native valve commissures, and
the upstream skirt applies a downstream axial force on an upstream side of the native valve complex.

15. The method according to claim 14, wherein providing the valve prosthesis comprises providing the valve prosthesis such that the axial support arches extend in an upstream and radially outward direction.

16. The method according to claim 14, wherein providing the valve prosthesis comprises providing the valve prosthesis such that the axial support arches extend radially outward in a direction orthogonal to a central longitudinal axis of the prosthesis.

17. The method according to claim 14, wherein positioning the valve prosthesis does not comprise rotationally aligning the valve prosthesis with respect to the native valve complex.

18. The method according to claim 14, wherein positioning the valve prosthesis comprises positioning the valve prosthesis such that the axial support arches protrude radially outward over the native leaflet tips.

19. The method according to claim 14, wherein positioning the valve prosthesis comprises positioning the valve prosthesis such that the axial support arches do not extend to floors of aortic sinuses of the native valve complex.

20. The method according to claim 14, wherein positioning the valve prosthesis comprises:
radially collapsing the support frame prior to delivery of the valve prosthesis to the native valve complex;
transluminally delivering the valve prosthesis to a vicinity of the native valve complex while the support frame is radially collapsed; and
radially expanding the support frame at the native valve complex.

21. The method according to claim 14, wherein positioning the valve prosthesis comprises positioning the valve prosthesis such that the upstream and downstream axial forces together anchor the valve prosthesis to the native valve complex.
